(19) United States
(12) Patent Application Publication
(10) Pub. No.: US 2024/0188835 A1
(43) Pub. Date: Jun. 13, 2024

(54) System and method for non-invasive assessment of cardiovascular and pulmonary murmurs

(71) Applicant: AventuSoft, LLC, Boca Raton, FL (US)

(72) Inventors: Claudia Iannini, Pembroke Pines, FL (US); Kaustubh Kale, Delray Beach, FL (US)

(73) Assignee: AventuSoft, LLC, Boca Raton, FL (US)

(21) Appl. No.: 18/444,684

(22) Filed: Feb. 17, 2024

Related U.S. Application Data
(63) Continuation-in-part of application No. 17/983,343, filed on Nov. 8, 2022.
Application No. 17/983,343 is a continuation-in-part of application No. 16/741,740, filed on Jan. 13, 2020, now Pat. No. 11,490,849.
Application No. 16/741,740 is a continuation-in-part of application No. 15/397,138, filed on Jan. 3, 2017, now Pat. No. 10,531,839.

(51) Int. Cl. A61B 5/0205; A61B 5/00; A61B 5/02; A61B 5/08; A61B 5/11; A61B 7/00

(52) U.S. Cl. CPC A61B 5/0205; A61B 5/02007; A61B 5/1102; A61B 5/7221; A61B 7/00; A61B 5/08

(57) ABSTRACT

A method or system for noninvasive evaluating and monitoring of abnormal cardiopulmonary vibrations includes obtaining one or more signals using one or more noninvasive heart and/or lung vibration signal sensors or transducers that provide a measure of physiological effects that are correlated with cardiopulmonary functions, recording, by a processor and one or more heart and/or lung vibration signal sensors or transducers for turbulence recordings between a first and second cardiopulmonary sounds, analyzing the turbulence recordings for unique vibration signatures associated with cardiopulmonary disease by searching deviations from an average beat, and presenting an assessment of cardiopulmonary disease upon finding that the deviations are above a predetermined threshold. In some embodiments, the system and method further include seeking a largest mean absolute error of a specific segment between an average beat and each beat from a subject to provide a murmur level indicative of cardiopulmonary disease.

### CROSS REFERENCE TO RELATED APPLICATIONS

This Application is a Continuation-in-Part of and claims priority through U.S. patent application Ser. No. 17/983,343 filed on Nov. 8, 2022 and a Continuation-in-part through U.S. patent application Ser. No. 16/741,740 filed on Jan. 13, 2020, which claims priority through U.S. patent application Ser. No. 15/397,138 filed on Jan. 3, 2017 which further claims the priority benefit of Provisional Application Nos. 62274766, 62274761, 62274763, 62274765, and 62274770 each of which were filed on Jan. 4, 2016, the entire disclosure of each are incorporated herein by reference.

### FIELD

The present application relates to systems and methods for noninvasive detection, evaluation and monitoring of diverse pathological cardiovascular and pulmonary murmurs, for example those acquired by the presence of valvular heart diseases, such as aortic stenosis (AS), mitral regurgitation (MR), aortic regurgitation (AR), mitral stenosis (MS), and mitral valve prolapse (MVP), or those due to impaired blood flow in the heart or cardiac vessels, such as the murmur generated by the presence of coronary artery disease (CAD), third sound (S3), and fourth sound (S4) among others, as well as those due to lung diseases or airflow obstructions, including chronic obstructive pulmonary disease (COPD).

### BACKGROUND

While stethoscopes are the preliminary method for assessing the presence of a heart murmur, an echocardiogram is currently the gold standard for assessing the valvular disease causing an observed heart murmur, such as aortic stenosis (AS). This is because echocardiograms show the blood flow through the heart and its valves, as well as the valves performance and whether issues are present preventing their proper function. Although generally safe, an echocardiogram is expensive (currently approximately $1000-$3000) and time consuming (20-60 minutes), and research indicates that some of the measures may not be reliable, e.g., in severe mitral regurgitation. The potential role of biomarkers in screening and diagnosing of heart murmurs has been the subject of increasing interest over the Brain natriuretic peptide (BNP) and N-terminal BNP (NT-proBNP) are two biological substances found in the blood which have been associated with the severity of many valvular heart diseases (AS, MR, AR, MS, etc.) and that correlate with echocardiographic markers of higher risk of adverse outcomes in AS. Other biomarkers currently under investigation for their role in valve diseases include troponin I, troponin T, acquired von Willebrand factor (vWF), among others. Blood tests may not be available at all facilities, and having blood drawn with a needle caries its own risks, including excessive bleeding, fainting, hematoma, and infection. Furthermore, common over-the-counter doses of biotin supplements interfere with assays for NT-proBNP, and usually patients are required to stop biotin consumption for at least 72 hours prior to the collection of a blood sample.

### SUMMARY

Given the impracticality, cost and availability of regular echocardiography assessments and lack of validated biomarkers for diagnosing and managing cardiovascular or pulmonary (or generally referred to hereafter as “cardiopulmonary”) murmurs, novel non-invasive point-of-care technologies can identify patients correctly, thereby further improving the safety and accuracy of a multifaceted approach to the initial diagnosis of cardiopulmonary murmurs. With the growing risk of murmurs in our population, a tool for providing noninvasive assessment of aortic stenosis and other cardiovascular and pulmonary murmurs is essential today.

The present application relates to systems and methods for noninvasive detection, evaluation and monitoring of diverse pathological cardiopulmonary murmurs, for example aortic stenosis (AS), mitral regurgitation (MR), aortic regurgitation (AR), mitral stenosis (MS), mitral valve prolapse (MVP), coronary heart disease (CAD), third sound (S3), fourth sound (S4), chronic obstructive pulmonary disease (COPD) etc., using at rest assessment of hemodynamic performance, based on quantitative measurements of heart and lung sound related parameters and cardiac events for diagnostic and therapeutic purposes, including obtaining one or a plurality of signals or data from one or a plurality of noninvasive sensors or transducers that measure one or a plurality of physiological effects that are correlated with cardiopulmonary functions; transmitting the data to a computing device having analysis software; using a trained algorithm to process the data to determine a presence and stage of the cardiovascular and pulmonary disease or heart murmurs, e.g., of AS; and generating an output indicative of the state or condition of the analysis.

There is a need for noninvasive cardiopulmonary health assessment and monitoring systems and methods that can provide timely and enhanced patient experience by enabling telehealth remote patient monitoring (RPM) or at-home self-monitoring of heart and lung conditions. There is also a need for an accurate and affordable screening tool that enables early identification and treatments of certain serious conditions and increase survival rate. Therefore, there is a need for systems and methods that can effectively measure and one assess or more different cardiopulmonary conditions. There is also a need to integrate such cardiopulmonary health assessment systems and methods with computing devices that continue to grow in computing capability and power.

The exemplary embodiments herein provide methods and systems based on a technique of separating, identifying, extracting, and marking cardiac events from noninvasively captured physiological signals related to hemodynamics and assess the cardiopulmonary health in a subject, and identifying the presence of cardiovascular and/or pulmonary pathological murmurs, such as but not limited to aortic stenosis (AS).

The features or parameters can be ultimately used in a trained model or classifier (e.g., a trained machine-learned classifier) to estimate metrics associated with the physiological state of a subject, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

The cardiohemic system models the kinematics of the motion of the lumped system which consists of the ventricle, its contents, and surrounding structures such as walls, valves, and blood, displaced by inertial forces, creating fluid deceleration in the ventricle and its causal relation to the oscillations (vibrations and rotational inertia) due to abrupt mechanical opening and closing of the valves during each cardiac cycle.

Examples of cardiac vibration objects are the first sound, the second sound, the third sound, the fourth sound, ejection sounds, opening sounds, murmurs, heart wall motions, coronary artery sounds, and valve sounds of the Mitral valve opening and closing, Aortic valve opening and closing, Pulmonary valve opening and closing, Tricuspid valve opening and closing. Examples of the pulmonary vibration objects are the respiratory lung sounds, breathing sounds, tracheobronchial sounds, vesicular sounds, Broncho vesicular sounds, or snoring sounds. A portion of the energy produced by these vibrations lies in the infra-sound range (generally below 20 Hz), which falls in the inaudible and low-sensitivity human hearing range. Another portion of the energy produced by these vibrations falls in the audible hearing range. Vibration transducers or accelerometers placed on the thoracic region or chest capture these vibrations from both of these ranges. Data can be obtained using a tri-axial accelerometer or multiple tri-axial accelerometers placed on different points of the torso.

Similarly, other sensor modalities capture signals correlated with the cardiohemic events, including but not limited to electrocardiogram (ECG), impedance cardiography (ICG), phonocardiography (PCG), photoplethysmography (PPG), seismocardiography (SCG), ballistocardiography (BCG), gyrocardiography (GCG), along with echocardiography (echo).

The opening and closing of the atrioventricular (AV) valves are dependent on pressure differences between the atria and ventricles. When the ventricles relax, atrial pressure exceeds ventricular pressure, the AV valves are pushed open and blood flows into the ventricles. Conversely, when the ventricles contract, ventricular pressure exceeds atrial pressure causing the AV valves to snap shut.

The semilunar valves (pulmonary valve and aortic valve) are one-way valves that separate the ventricles from major arteries. The aortic valve separates the left ventricle from the aorta, while the pulmonary valve separates the right ventricle from the pulmonary artery. As the ventricles contract, ventricular pressure exceeds arterial pressure, the semilunar valves open and blood is pumped into the major arteries. Conversely, when the ventricles relax, arterial pressure exceeds ventricular pressure and the semilunar valves snap shut. This is due to the elevated pressures in the aorta and the pulmonary artery pushing the blood back toward the ventricles to close the semilunar valves.

By analyzing one or a plurality of the physiological signals capture noninvasively, including, but are not limited to ECG, ICG, PCG, SCG, BCG, GCG, PPG, and echo, the vibration present in each heartbeat can be detected, quantified and categorized into normal or one having abnormal heart murmurs, depending on its location and type, e.g., a high-pitched, diamond shaped crescendo-decrescendo vibration during systole is characteristic of Aortic Stenosis (AS).

In some embodiments, a method for noninvasive evaluating and monitoring of cardiopulmonary disease based on quantitative measurements of cardiac events for diagnostic and therapeutic purposes can include obtaining one or more signals using one or more noninvasive heart vibration signal sensors or transducers that provide a measure of one or more physiological effects that are correlated with cardiopulmonary functions and recording, by at least one processor and the one or more heart vibration signal sensors or transducers for turbulence recordings between a first and a second cardiac sounds. The method can further include analyzing the turbulence recordings between the first and the second cardiac sounds for unique vibration signatures associated with valve disease by searching for one or more deviations from an average beat and presenting an assessment of valve disease upon finding that the one or more deviations are above a predetermined threshold.

In some embodiments, the method further includes the step of seeking a largest mean absolute error of a specific segment between an average beat and each beat from a subject to provide a heart murmur level indicative of valve disease.

In some embodiments, the one or more noninvasive heart vibration signal sensors or transducers are one or more noninvasive seismocardiography (SCG) heart vibration sensors or transducers.

In some embodiments, the valve disease evaluated and monitored is one among a number of pathological cardiac murmurs such as aortic stenosis. In other embodiments, the valve disease evaluated can be one or more among aortic stenosis (AS), mitral regurgitation (MR), aortic regurgitation (AR), mitral stenosis (MS), mitral valve prolapse (MVP), coronary artery disease (CAD), S3 sound, S4 sound, or chronic obstructive pulmonary disease (COPD) among others.

In some embodiments, the first heart sound is S1 and the second heart sound is S2. In yet other embodiments, the first heart sound is S2 and the second heart sound is S3. In various embodiments, the first heart sound can be a first valvular event and the second heart sound can be a second valvular event and not just limited to S1, S2, S3, S4, etc.

In some embodiments, the method can include analyzing the noise recordings between the first and the second cardiac sounds for unique vibration signatures associated with valve disease is done by searching for the one or more deviations from the average beat during a selected segment such as a late systole segment or a late diastole segment.

In some embodiments, a system for noninvasive evaluating and monitoring of cardiopulmonary disease based on quantitative measurements of cardiac events for diagnostic and therapeutic purposes can include at least one or more non-invasive vibration signal sensors or transducers for capturing one or more cardiac waveform signals providing a measure of one or more physiological effects that are correlated with cardiopulmonary functions, one or more processors operatively coupled to the at least one or more non-invasive vibration signal sensors or transducers, memory having computer instructions and coupled to the one or more processors. The computer instructions when executed by the one or more processors causes the system to perform the operations of obtaining one or more signals using the one or more noninvasive heart vibration signal sensors or transducers that provide the measure of one or more physiological effects that are correlated with cardiopulmonary functions and recording, by the at least one or more processors and the one or more heart vibration signal sensors or transducers for turbulence recordings between a first and a second cardiac sounds. The system can include computer instructions causing the system to analyze the turbulence recordings between the first and the second cardiac sounds for unique vibration signatures associated with cardiopulmonary disease by searching for deviations from an average beat and present an assessment of cardiopulmonary disease upon finding that the one or more deviations are above a predetermined threshold.

In some embodiments, the system further includes the step of seeking a largest mean absolute error of a specific segment between an average beat and each beat from a subject to provide a heart murmur level indicative of valve disease.

In some embodiments the one or more noninvasive vibration signal sensors or transducers are an electrocardiogram (ECG) noninvasive a sensor and one or more sensors among seismocardiography (SCG) sensor, a gyrocardiography (GCG) sensor, impedance cardiography (ICG) sensor, a phonocardiophy (PCG) an sensor, a photoplethysmogram (PPG) sensor, or a ballistocardiogram (BCG) sensor.

In some embodiments, the system can include analyzing the noise recordings between the first and the second cardiac sounds for unique vibration signatures associated with valve disease is done by searching for the one or more deviations from the average beat during a selected segment such as a late systole segment or a late diastole segment.

In some embodiments, an apparatus for noninvasive evaluating and monitoring of cardiopulmonary disease based on quantitative measurements of cardiac events for diagnostic and therapeutic purposes can include at least two or more non-invasive vibration signal sensors or transducers for capturing one or more cardiac waveform signals providing a measure of one or more physiological effects that are correlated with cardiopulmonary functions using cardiac timing intervals, one or more processors operatively coupled to the at least one or more non-invasive vibration signal sensors or transducers, and memory having computer instructions and coupled to the one or more processors. The computer instructions cause the system to perform the operations of obtaining one or more signals using the one or more noninvasive heart vibration signal sensors or transducers that provide the measure of one or more physiological effects that are correlated with cardiopulmonary functions, recording, by the at least one or more processors and the two or more heart vibration signal sensors or transducers for turbulence recordings between a first and a second cardiac sound, analyzing the turbulence recordings between the first and the second cardiac sounds for unique vibration signatures associated with valve disease by searching for deviations from an average beat, and presenting an assessment of valve disease upon finding that the one or more deviations are above a predetermined threshold.

In some embodiments, a first non-invasive vibration signal sensor is an electrocardiogram sensors and wherein a second non-invasive vibration signal sensor or transducer is one among a seismocardiography (SCG) sensor, a gyrocardiography (GCG) sensor, an impedance cardiography (ICG) sensor, a phonocardiophy (PCG) sensor, a photoplethysmogram (PPG) sensor, or a ballistocardiogram (BCG) sensor.

### DESCRIPTION OF THE DRAWINGS

The accompanying drawings illustrate the embodiments of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 illustrates heart sounds signals captured on the chest representing different heart valve diseases (VHDs);

FIG. 2 illustrates a cardiac cycle in relation with various cardiac time intervals (CTIs);

FIG. 3 illustrates a system for the extraction, identification, marking and display of the noninvasive cardiohemic signals in accordance with one embodiment;

FIG. 4 is a flowchart of a telehealth Remote Patient Monitoring (RPM) System in accordance with one embodiment;

FIG. 5 illustrates the data recorded with a device using the system, apparatus, and methods herein (the apparatus also known as “HEMOTAG”) on all 4 channels for a patient with no aortic stenosis in accordance with the embodiments;

FIG. 6 illustrates the data recorded with the HEMOTAG of a patient with severe aortic stenosis in accordance with the embodiments;

FIG. 7 illustrates the beat-wise mean absolute deviation and Heart Murmur Level (HML) for both a no stenosis patient (in gray or lighter lines) and a patient with severe aortic stenosis (in darker lines) in accordance with the embodiments;

FIG. 8A illustrates data recorded on a healthy patient with a noise artifact in the zone of interest in accordance with the embodiments;

FIG. 8B illustrates the data recorded on the patient of FIG. 8A after removal of the noise artifact in accordance with the embodiments;

FIG. 9A shows the performance chart of the heart murmur level (HML) subdivided according to aortic stenosis severity; and

FIG. 9B shows the performance chart of the heart murmur level (HML) divided into two subgroups: innocent (no and mild aortic stenosis levels) vs structural (moderate and severe aortic stenosis levels).

### DETAILED DESCRIPTION

The present disclosure is best understood with reference to the detailed figures and description set forth herein. In the following description, various embodiments will be described. For the purpose of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

The described embodiments may be implemented manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by practitioners of the art to which the embodiments pertain. Persons skilled in the art will envision many other variations that are within the scope of the claimed subject matter.

The exemplary embodiments are directed to a system and method for non-invasively determining at least one of the potentially present abnormal murmurs in a subject's cardiopulmonary system.

The present subject matter is based on the fact that a healthy cardiopulmonary system in the human body represents a delicate coupling between heart pumping characteristics, valves openings and closings, fluid volume status, and filling pressures. The optimum coupling is impaired by aging, arterial and cardiovascular and pulmonary diseases, such as hypertension, heart failure, chronic obstructive pulmonary disease (COPD), coronary artery disease, and various heart valve diseases.

Hemodynamic evaluation is an essential component in diagnosing cardiovascular disorders and managing patient care.

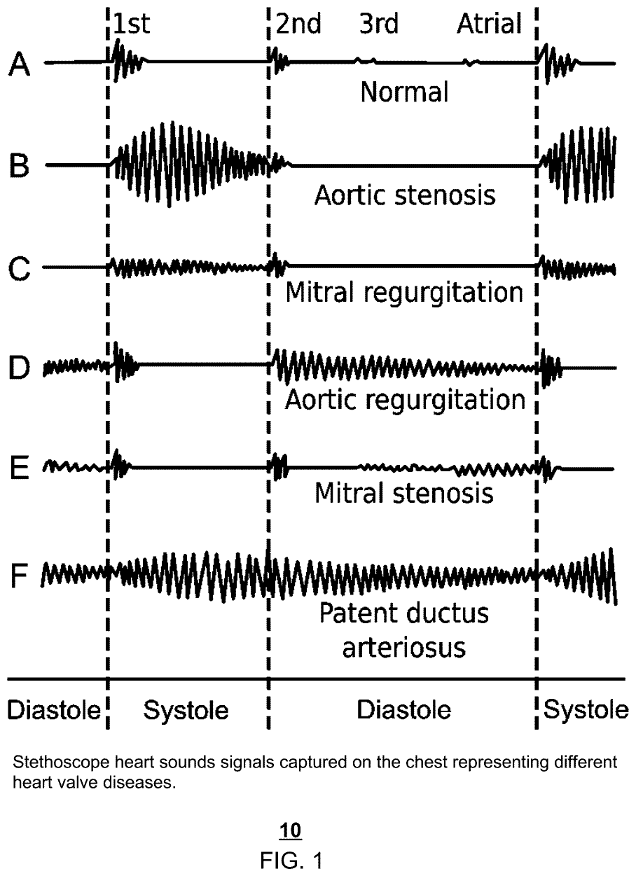

As the seismocardiogram (SCG) is captured, many types of heart murmurs can be identified depending on their appearance and location in the heartbeat, as shown in in the representation 10 of FIG. 1.

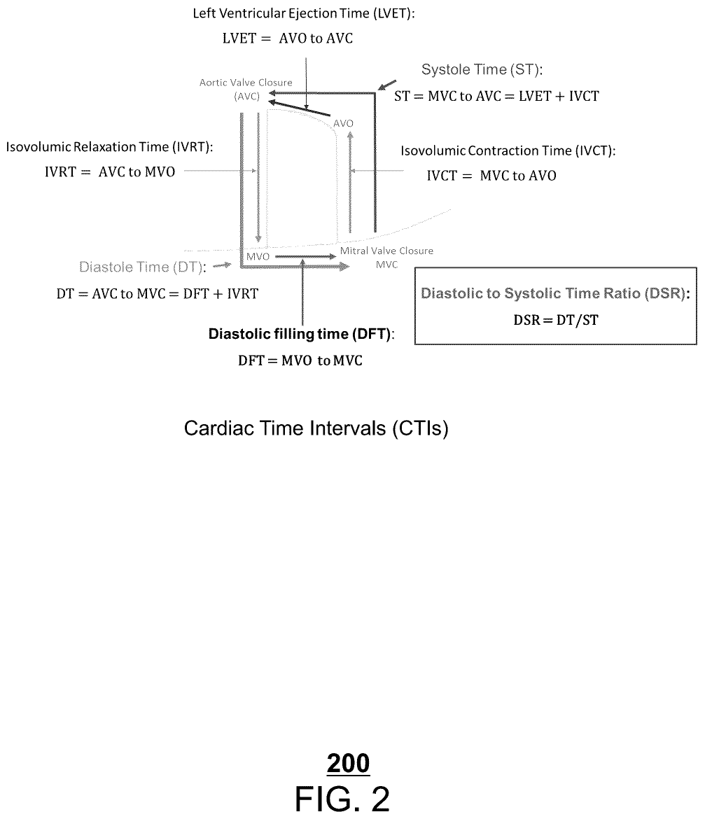

The cardiac time intervals (CTIs) are shown in the representation 200 of FIG. 2.

The slope of the end-systolic pressure-volume relationship (ESPVR) indicates end-systolic elastance, an index of contractility; and the slope of the end-diastolic pressure-volume relationship (EDPVR) indicates ventricular elastance or stiffness (the reciprocal of ventricular compliance). Several physiological relevant hemodynamic parameters can be determined from this, specifically the cardiac events corresponding to the opening and closing of the heart valves: Mitral Valve Closing (MVC), Mitral Valve Opening (MVO), Aortic Valve Opening (AVO) and Aortic Valve Closing (AVC). As the pressure increases in the ventricle as the aortic valve is open, the blood accelerates in the aorta resulting in the Rapid Ejection (RE) peak. Ventricular filling occurs along the EDPVR, a monotonically increasing curve: pressure is directly proportional to the volume of blood in the left ventricle, and volume is proportional to filling time.

Under normal conditions (shown by the top phonocardiogram (PCG) in FIG. 1), the heartbeat should not show any sort of abnormal vibration other than the ones caused by the blood pumping through the heart valves, which are opening and closing. There are a total of two normal cardiac sounds: the closing of the mitral and tricuspid valves and the opening of the aortic valve generate the vibrations typical of the first cardiac sound (S1), while the closing of the aortic and pulmonic valves and the opening of the mitral valve produce the characteristic vibrations of the second cardiac sound (S2). In patients with cardiac murmurs (the other 5 examples shown in FIG. 1), vibrations will be introduced and recorded, as the cardiovascular system is impaired, e.g., the heart valves are narrower or not properly closing. Vibrations will also be introduced and recorded in patients with pulmonary diseases, such as chronic obstructive pulmonary disease (COPD).

Consequently, as the frequency of the recorded murmur increases as the severity of the disease increases, so does the Heart Murmur Level (HML). Thus, the HML provides an alternative method, for accurate and absolute estimation of presence and severity of heart murmurs, such as AS, MR, MVP, MS, S3, S4, CAD etc., to that of using an echocardiogram.

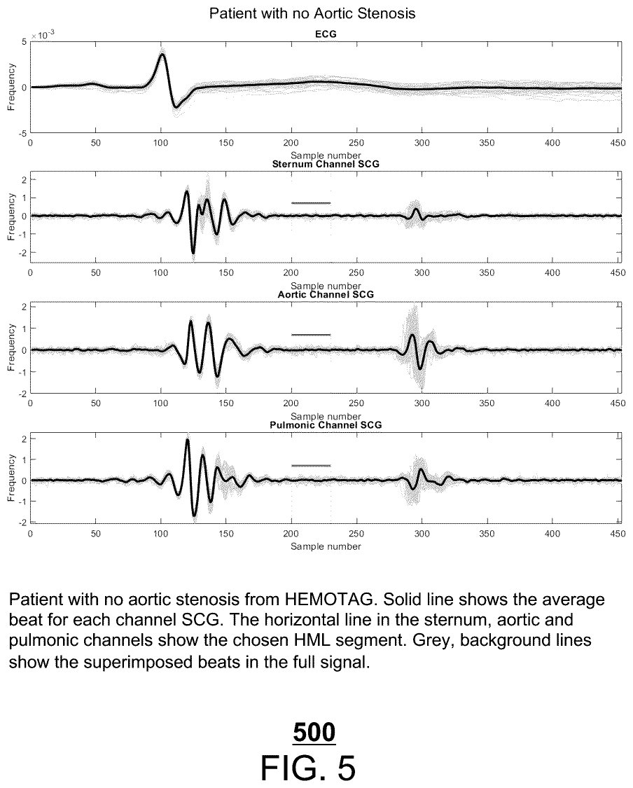

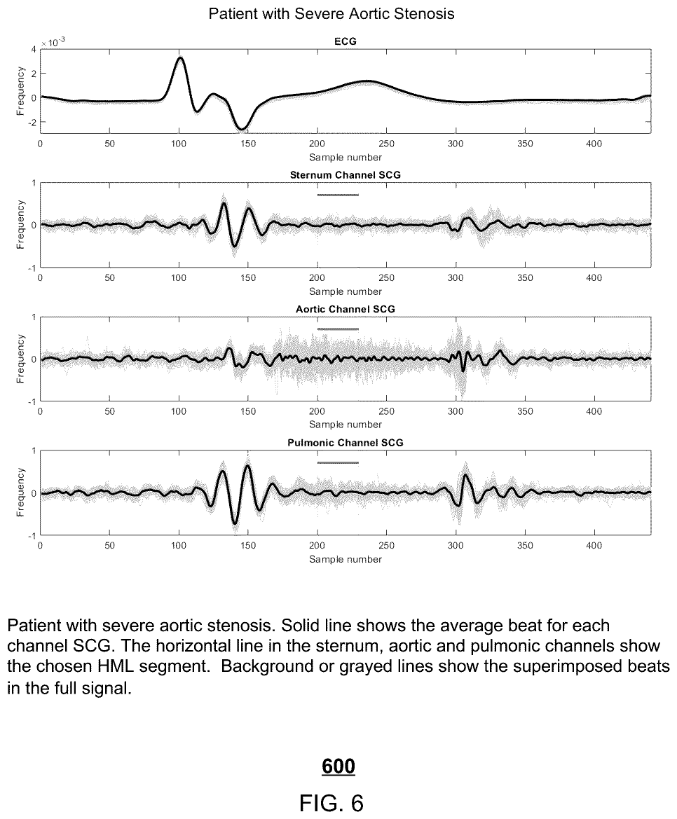

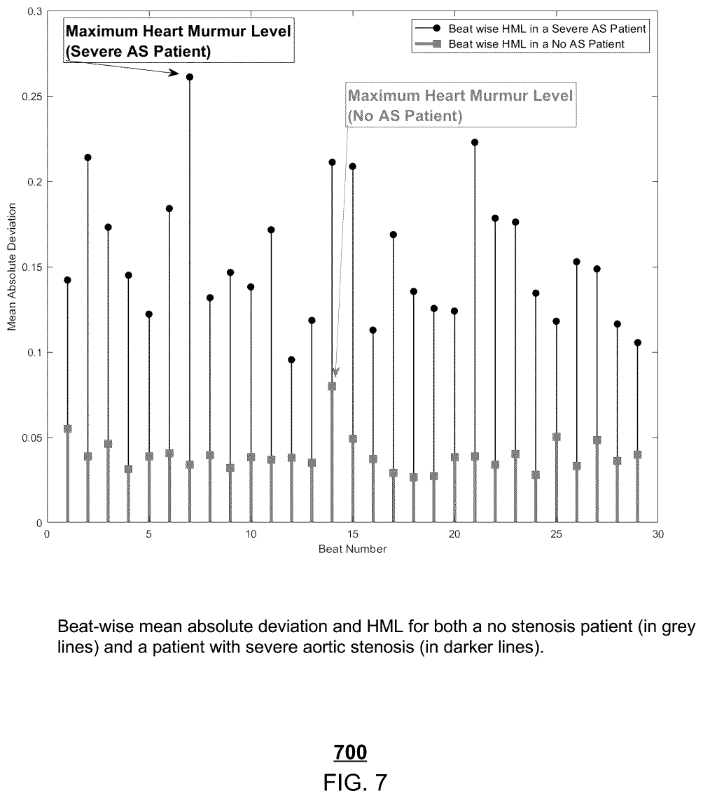

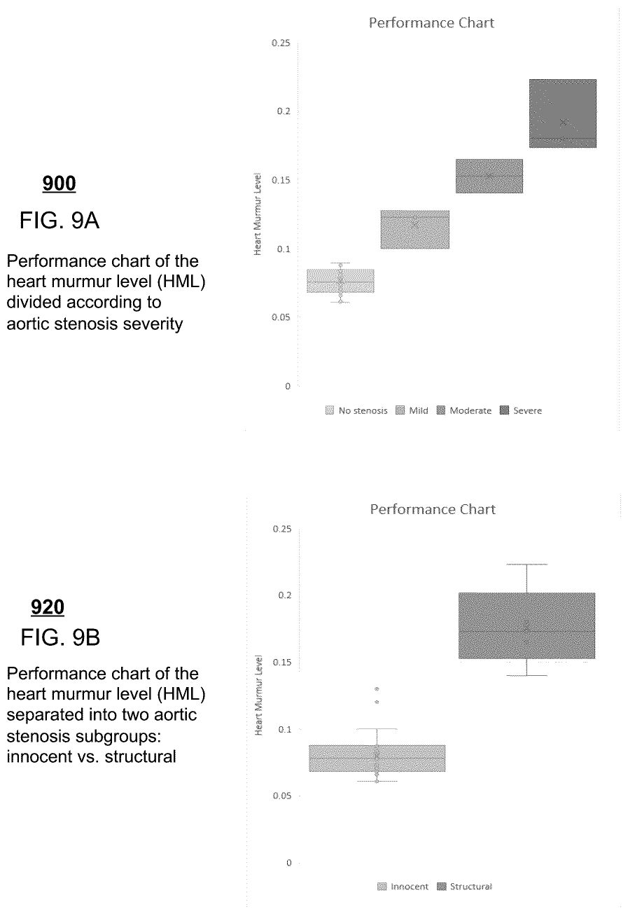

Under normal conditions (shown by the top phonocardiogram (PCG) in FIG. 1), the heartbeat should not have a significant HML. In patients with cardiac murmurs (the other 5 examples shown in FIG. 1), the HML will be higher to indicate the presence of abnormal vibrations. To determine the HML, the recorded cardiac data is segmented into the corresponding heart beats, based on the R peaks of the simultaneous ECG, from which the average beat is obtained (the darker, solid lines in FIGS. 5 and 6). The mean absolute error (MAE) between each beat and the average beat was calculated during a specific segment, and the maximum MAE is then recorded as the Heart Murmur Level or HML (in FIG. 7, the beat wise mean absolute differences and the HMLs for a patient with no AS (in grey) and a patient with severe AS (in darker lines) are shown). Based on the HML threshold, patients can then be categorized into the correct stage of the cardiac murmur tested, e.g., no AS, AS mild, moderate or severe (FIG. 9A). Patients can also be divided into two or more other subgroups for analysis based on their HML threshold, e.g., the innocent group, which can include the no murmur and mild stage, and the structural group, which can include the moderate and severe stages (FIG. 9B).

In another embodiment, the other common cardiac time intervals (CTIs) associated with the four valve events are (shown in FIG. 2):

Left Ventricular Ejection Time:(LVET)≙AVO to AVC

Isovolumic Contraction Time:(IVCT)≙MVC to AVO

Isovolumic Relaxation Time:(IVRT)≙AVC to MVO

Diastolic time:DT≙(AVC to MVC)

Systolic time:ST≙(MVC to AVC)

and the parameters can be combined to form derivative CTIs or ratios directly or normalized by the heart rate (HR) in beats per minute.

In another embodiment, a section of the systole phase, such as the mid- to late systolic segment (LSS), is another key index for identifying the presence of systolic heart murmurs, such as aortic stenosis and mitral regurgitation, and defined as:

LSS≙(RE to AVC).

For AS patients (shown by the second PCG from the top in FIG. 1), the aortic valve is narrowed or thickened due to valve calcification, therefore not able to fully open, resulting in a reduction or blockage of blood flow from the heart to the aorta and the rest of the body. This blockage causes the heart to pump harder to get enough blood to go through the body, therefore leading to a thickening and enlargement of the left ventricle. While the duration of the left ventricular systole is not prolonged in aortic stenosis, the higher level of left ventricular pressure due to the late closure of the aortic valve prolongs the left ventricular ejection, thus resulting in the crescendo-decrescendo systolic vibration that can be observed in between the first and second sound. In a patient with severe AS, an S4 can be audible.

In MR patients (shown by the third PCG from the top in FIG. 1), the mitral valve displays incompetent leaflets that may droop or bulge, thus not allowing the valve to properly close. This causes a backflow from the left ventricle into the left atrium. This murmur usually begins after S1 and continues to the second hard sound (S2), with an intensity that may vary.

Considered a systolic click-murmur syndrome as it can lead to MR, mitral valve prolapse (MVP) displays a billowing of the mitral valve leaflets into the left atrium during systole. This implies that the mitral valve flaps bulge backwards into the left atrium as the heart contracts, thus possibly causing blood to leak backwards into the chamber. This murmur produces a mid-systolic click usually followed by a uniform, high-pitched vibration during late systole, which is actually due to the MR that typically comes with the MVP.

In another embodiment, a section of the diastole phase, such as the mid- to late diastolic segment (LDS), is another key index for identifying the presence of diastolic heart murmurs, such as aortic regurgitation and mitral stenosis, and defined as:

LDS≙(MVO to MVC).

In AR patients (shown by the fourth PCG from the top in FIG. 1), the aortic valve displays incompetent leaflets, which cause a backward flow from the aorta into the left ventricle during diastole, as the left ventricle relaxes. The murmur caused by AR is blowing, high-pitched, diastolic, and decrescendo, beginning soon after the aortic component of S2. If AR is slight, the murmur may occur only in early diastole. If left ventricular (LV) diastolic pressure is very high, the murmur is short because aortic and left ventricular diastolic pressures equalize earlier in diastole.

In MS patients (shown by the fifth PCG from the top in FIG. 1), the mitral valve leaflets become thickened and immobile most often due to rheumatic fever, which can cause the valve to thicken with scar tissue, therefore narrowing the mitral orifice, which in turn prevents the blood to easily flow from the left atrium to the left ventricle. Upon auscultation, mitral stenosis includes a loud S1, due to the leaflets closing abruptly (MVC), and a low-pitched decrescendo-crescendo rumbling diastolic vibration.

In another embodiment, the current method may be extended to valvular heart diseases that affect the right side of the heart, specifically those affecting the pulmonic and the tricuspid valves. These may be but are not limited to pulmonic stenosis (PS), pulmonic regurgitation (PR), tricuspid stenosis (TS), and tricuspid regurgitation (TR).

In another embodiment, the diastole phase is another key index for identifying the presence of a third (S3) and/or a fourth (S4) cardiac sound.

The third heart sound (S3), also called ventricular gallop, is a low-frequency, short vibration occurring in early diastole just after S2 when the mitral valve opens. It is produced during passive LV filling when a large amount of blood strikes a compliant left ventricle. While it may be a normal finding in pregnant women, children, and well-trained athletes, it may also be a sign of systolic heart failure, as the overly compliant myocardium can result in a dilated left ventricle.

The fourth heart sound (S4), also called atrial gallop, is a low-pitched vibration occurring in late diastole, just before S1 when atrial contraction forces blood into a noncompliant left ventricle. It is rarely a normal finding as it in fact is most often a sign of diastolic heart failure or active ischemia, resulting in an impaired relaxation of the left ventricle. Note that cardiac or heart sounds as referenced in the claims and elsewhere in the specification are not necessarily limited to the heart sounds S1, S2, S3, or S4 referenced above.

In another embodiment, the current method may be extended and implemented to assess and monitor other cardiac murmurs produced by cardiovascular diseases that limit blood flow to the heart, such as but not limited to coronary artery disease (CAD). Detection and monitoring of such sounds can be done by implementing one or more signal processing technique, including but not limited to the Fast Fourier Transform method, the Autoregressive method, the Autoregressive Moving Average method, and the Minimum-Norm (Eigen-vector) method.

In CAD patients, the vessels that supply blood to the heart have become narrowed or blocked, due to the buildup of fatty deposits, cholesterol and other substances known as plaque on their inner walls. This condition thus limits the blood flow to the heart, leading to various complications, including but not limited to myocardial infarction. While the leading cause of this condition is atherosclerosis, other risk factors include smoking, diabetes, high cholesterol levels, and high blood pressure among others. While in a healthy patient, the coronary blood flow is maximum during diastole, in a patient with CAD the blood flow will be turbulent, due to the blockage or narrowing of the vessels. This will therefore produce a faint turbulence or soft murmur that can be detected.

In another embodiment, the current method may be extended and implemented to assess and monitor pulmonary murmurs, such as but not limited to the ones associated with pulmonary conditions, such as chronic obstructive pulmonary disease (COPD). Such conditions also leave patterns that can be analyzed and identified using the techniques disclosed herein.

In COPD patients, the airways and other parts of the lungs are damaged, thus blocking the airflow making it hard to breathe. This condition is typically due to emphysema and chronic bronchitis. In the first case, the walls between the air sacs in the lungs are damaged, therefore not properly inflating and deflating as air travels through the lungs. In the second case, the linings of the airways are irritated and inflamed, which forms mucus in the lung, which results in difficulty breathing. COPD can cause a variety of lung sounds, including rhonchi, wheezing, and crackling. Rhonchi are due to a buildup of secretions in the upper airways and are low pitched, continuous gurgling sounds that can be heard during inspiration or expiration. Wheezing sounds are due to the vibrations of the narrowed walls of small airways, and it manifests as high-pitched whistling sound during expiration. Crackling noises stem from air bubbles passing through fluid (typically mucus) in the airways, and is usually prolonged and low pitched, although it can be high pitched in some cases.

Additionally or alternatively, to distinguish preserved vs. impaired LV systolic function, the systolic time ratio (STR) can be used, computed using the following CTIs:

STR≙AVO/(AVO to AVC)=PEP/LVET

where the pre-ejection period (PEP) is equivalent to the AVO with its starting point referenced at the onset of the QRS complex. To account for changes in heart rate (HR), a heart rate normalized, systolic time ratio index (STRi) can be used.

In another embodiment, the diastolic to systolic duration ratio (D/S ratio or DSR) is another key index for identifying diastolic heart failure and defined as:

DSR≙(AVC to MVC)/(MVC to AVC).

In an embodiment, to furthermore distinguish between diastolic and systolic dysfunction, both DSR and STR can be used.

In an embodiment, the CTI based features such as DSR and STR can be accurately obtained inexpensively and noninvasive using a single or a plurality of sensor modalities (which include but not limited to one or more sound transducers, accelerometers, gyroscopes, bioreactance sensors, and ECG electrodes) in any combination of physiological signals such as impedance cardiography (ICG), phonocardiography (PCG), seismocardiography (SCG), gyrocardiography (GCG), electrocardiogram (ECG), and as well as noninvasive measurements of the opening and closure of the aortic and mitral valves using different ultrasound modalities, i.e., echocardiogram (echo), such as M-mode, Doppler flow imaging, Tissue Doppler Imaging (TDI) or speckle tracking strains.

In an embodiment, AVC can be determined noninvasively using heart sounds either obtained from an acoustic sensor such as a digital stethoscope, i.e., PCG, additionally or alternatively from an accelerometer which measures the cardiac mechanical processes including cardiac muscle contraction, cardiac valve movement, blood flow turbulence, and momentum changes, i.e. SCG, or from angular displacement using gyroscope, i.e., GCG.

In an embodiment, the second heart sound (S2) consists of two major components: AVC or A2 and pulmonic valve closure (P2). The peak of the S2 envelope correlates to the onset of the aortic valve closure in a stable and consistent mechanical fashion, i.e., the S2 is caused by the AVC. This relationship can be established from training data consisting of paired S2 peak timing and AVC timing obtained from the gold standard echocardiography and is generalizable across patients for the same sensor modality. Manufacturing differences in sensors result in different filtering delays that require recalibration, i.e., the specific relationship is dependent of the sensor modality and settings. In one specific embodiment, a simple relationship is established using linear regression.

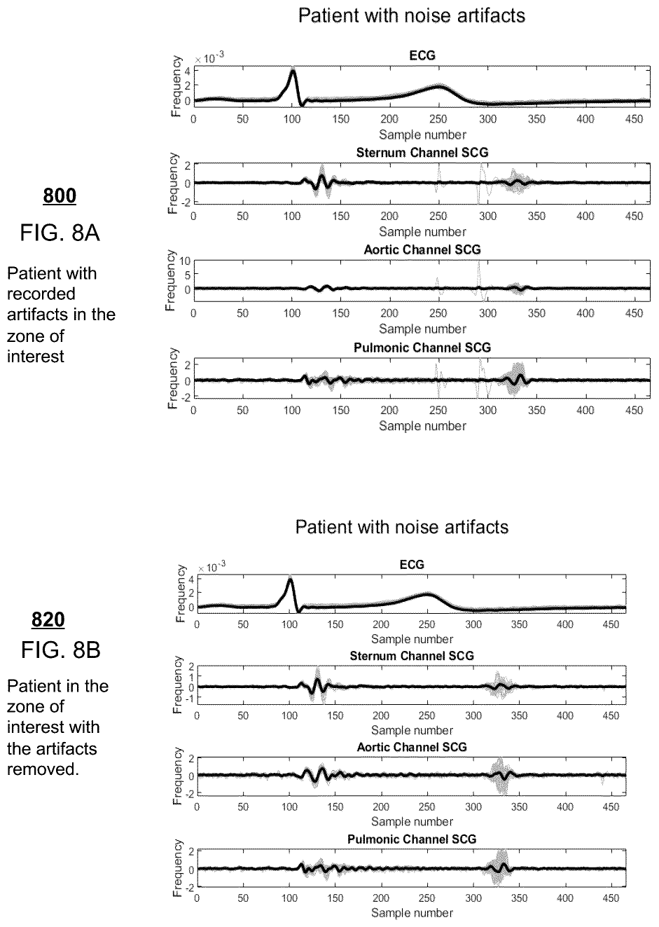

In another embodiment, the noise artifacts introduced in the data due to bodily functions other than the ones of interest or to external factors may be removed as they can negatively influence the heart murmur level (HML) results, as can be appreciated in FIGS. 8A and 8B. The system and method proposed here include a preprocessing step (noise removal, etc.), a data standardization that brings the preprocessed data to a common format (normalization, scaling, windowing, etc.).

In an embodiment, the method can be used to monitor one or more diagnosed cardiopulmonary disease and the correspondent progression thereof. While the cardiopulmonary disease (CPD) (s) may be mild at the time of diagnosis, and therefore may not require surgery or any type of intervention at that time, deterioration may happen, causing severe damage to the patient's heart, lungs, and health, including death. The method can therefore help in the monitoring of CPD (s) and alert domain experts (e.g., physicians, clinicians, etc.) to act in the case of murmur progression.

The exemplary embodiments herein provide a method and system based on a technique to identify the separated cardiopulmonary signals, to extract information contained in vibration objects.

In one embodiment, known under machine learning, auditory scene analysis or spare coding approach to the source separation problem. Data is obtained using a tri-axial accelerometer or multiple tri-axial accelerometers placed on different points of torso.

Source identification analysis in accordance with the methods described herein identify individual vibration objects described above from the source separated vibration signals. The individual vibration signals are identified to be from the mitral valve, aortic valve, tricuspid valve, the pulmonary valve, coronary artery, murmurs, third sound, fourth sound, respiratory sound, breathing, and snoring during individual heart beats.

The embodiments can include different source identification techniques specifically used for tagging the individual cardiopulmonary signals for application in a non-linear time variant system, such as Principal component analysis, Gabor filtering, Generalized Cross Correlation (GCC), Phase transform (PHAT), ROTH, SCOT and Band Filtering. Using 1) Spectral information, 2) Relations among channels, and 3) Relations among events in the form of relative times of occurrence.

In an alternative embodiment, applying machine learning techniques, such as a neural network of a single or multiple layers may be used to output the AVC from either S2 peak timing or directly from the raw recording. Note that the embodiments herein are not limited to the sensor modalities or methods used to obtain the CTIs and all CTI derived features such as LVET, STR, DSR, IVRT, LVET, IVCT, from noninvasive physiological signals.

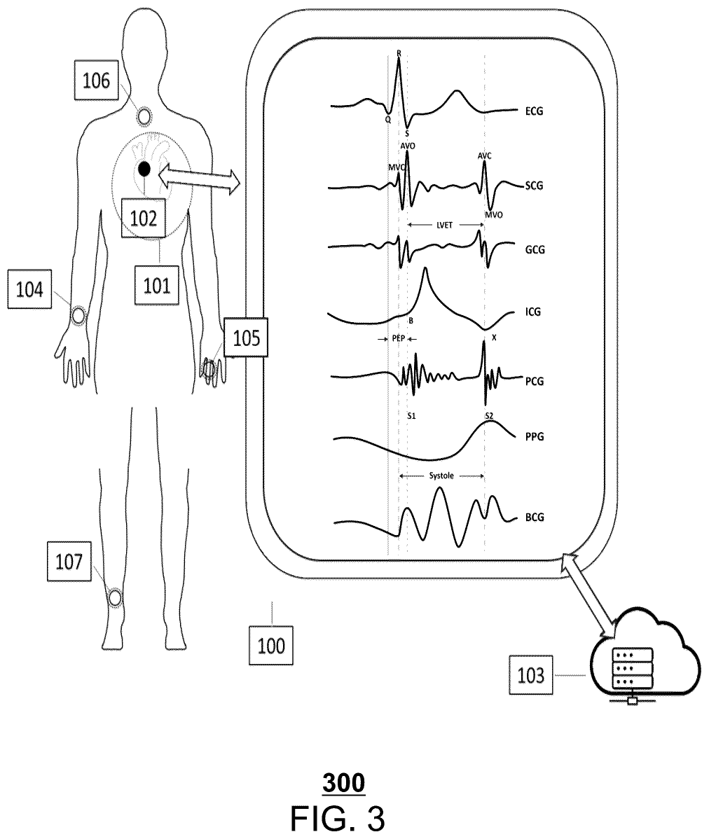

In an embodiment, to find the S2 envelope peak, we first segment cardiac signal into individual beats using synchronized electrocardiogram (ECG) recording and its fiducial points (FIG. 3). For each beat, the ECG and PCG (or SCG) are aligned using the onset of the QRS complex or u-point. The vibrational waveform (PCG or SCG) is bandpass filtered to emphasize the high frequency components, e.g., 50-240 Hz, then its energy envelope is used to find the peak amplitude and its corresponding time stamp. To further narrow the search window for potential S2 sound, we can leverage physiological knowledge, i.e., S2 occurs past the S-point of the QRS complex, around the T-wave, and marks the end of systole or the beginning of diastole. The diastole is the longer of the two phases, so that the heart can rest between contractions. We can use the beat duration (computed from R-R intervals) to mark the potential range of the two phases in each beat.

The exemplary embodiments of the system and method proposed here are shown in FIG. 3. The system 300 is an embedded platform 100 which can be any smart processing platform with digital signal processing capabilities, application processor, data storage, display, input modality like touch-screen or keypad, microphones, speaker, Bluetooth, and connection to the internet via WAN, Wi-Fi, Ethernet or USB. This embodies custom embedded hardware, smartphone, iPad-like and iPod-like devices. Area 101 in FIG. 3 is the auditory scene at the chest locations. Module 102 in FIG. 3 can include the transducer or transducer array used to capture the heart signal and the wearable microprocessor hardware with digital signal processing capabilities, application processor, Analog to digital frontend, data storage, input modality like buttons, and wireless connection via Bluetooth, Bluetooth low energy, near field communication transceiver, Wi-Fi, Ethernet or USB. Module 102 in FIG. 3 can also include the processor or signal processing module on the wearable that captures synchronized sensor data from the transducer array. The module 102 can save the data to memory and can communicate with platform 100 for data transfer. 100 in FIG. 3 is the module that allows a user to visually see the individual streams and information from the heart valves, in an embodiment this could be a connected display or any other modality of display. Module 102 can include multiple sensor transducers that capture the composite signal; comprising of the electrocardiogram signals, heart sounds, lung sounds and snoring sounds. Module 102 can be the wearable hardware that synchronously collects the signals across the transducers and is responsible for the analog to digital conversation, storage and transmission to the portable unit. Module 103 in FIG. 3 can be a cloud computing module that calculates vital sign from the input sensor stream coming from 102 for the Heart rate, breathing rate, EKG signal, skin temperature, and associated vitals. It encrypts the raw sensor data for transmission to the cloud computing module 103. It can also communicate with a dashboard on module 103 for data exchange, login, alerts, notifications, display of processed data. Module 103 in FIG. 3 can also be the cloud module that processes the individual streams for eventual source separation, identification and marking of the heart valve signals. Note however, contemplated embodiments herein can also have such processes done on-board at either the module 102 or module 100 given adequate computing and processing power.

The exemplary embodiments of the system and method proposed here include a portable electronic device or an embedded hardware system and a plurality of electronic components, the main elements required to capture body cardiohemic signals are the sensor units that capture, digitize, and process signals for noise reduction, filtering, and amplification.

The exemplary embodiments of the system and method proposed here provide a microcontroller that transmits the electrophysiological data received from the plurality of ECG electrodes, the sound transducer, and the vibrational and rotational sensors to at least one of the portable electronic device (PED) and a computing device. The PED and computing device are configured to: receive, in one or more temporal windows, a representation of data from one or more of the following when positioned against the thoracic cavity of the user: one or a plurality of ECG, ICG, PCG, SCG, BCG, GCG, PPG, and echo; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the cardiac or pulmonary health of the user and/or estimate a progression of a cardiac or pulmonary health condition.

For the exemplary embodiments of the system and method proposed here, the PED may be held against the thoracic cage of the user, the back of the user, and/or the sternum of the user to emit sound into the body of the user.

For the exemplary embodiments of the system and method proposed here may be a wearable, include, but not limited to a patch, ring, belt, band, bracelet, necklace, or clothing for prolonged capturing, monitoring, and transmission of electrophysiological data. 104-107 in FIG. 3 illustrates possible locations of device placement.

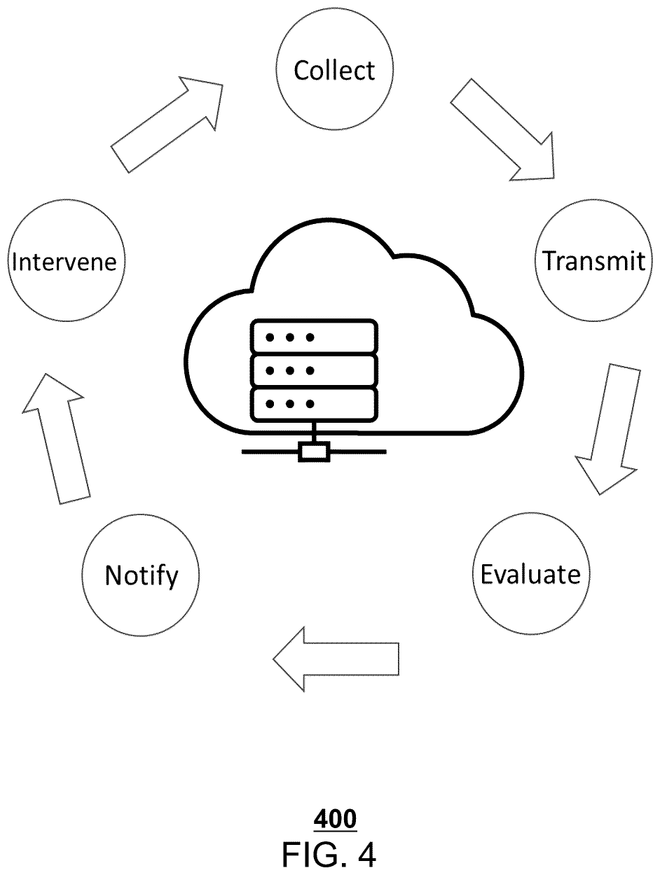

FIG. 4 is a flowchart of a telehealth Remote Patient Monitoring (RPM) System 400 in accordance with one embodiment where data is collected and transmitted for evaluation. The transmission and evaluation can be done in a cloud computing environment, but the contemplated embodiments are not necessarily limited to such environments. Based on such evaluation, appropriate notification can be provided to the care provider and/or patient enabling intervention and further collection and monitoring as illustrated.

FIG. 5 illustrates a representation 500 of the data recorded with a device using the system, apparatus, and methods herein (the apparatus also known as “HEMOTAG”) on all 4 channels for a patient with no aortic stenosis in accordance with the embodiments. The solid line shows the average beat for each channel SCG. The short horizontal line in each of the sternum, aortic and pulmonic channels show the chosen HML segment. The gray background lines show the superimposed beats in the full signal.

FIG. 6 illustrates a representation 600 where the data recorded with the HEMOTAG of a patient with severe aortic stenosis in accordance with the embodiments. The solid line shows the average beat for each channel SCG. The short horizontal line in each of the sternum, aortic and pulmonic channels show the chosen HML segment. Background or grayed lines show the superimposed beats in the full signal.

FIG. 7 illustrates a representation 700 where the beat-wise mean absolute deviation and Heart Murmur Level (HML) for both a no stenosis patient (in gray or lighter lines) and a patient with severe aortic stenosis (in darker lines) are shown in accordance with the embodiments.

FIG. 8A illustrates a representation 800 having data recorded on a healthy patient with a noise artifact in the zone of interest in accordance with the embodiments.

FIG. 8B illustrates a representation 820 of the patient of FIG. 8A after removal of the noise artifact in accordance with the embodiments.

FIG. 9A shows the performance chart 900 of the heart murmur level (HML) subdivided according to aortic stenosis severity and FIG. 9B shows the performance chart 920 of the heart murmur level (HML) divided into two subgroups: innocent (no and mild aortic stenosis levels) vs structural (moderate and severe aortic stenosis levels).

For the exemplary embodiments of the system and method proposed here, other noninvasively obtained biological information are used to supplement the captured signal such as age, height, weight, and sex.

Biological sex plays an important role in cardiac physiology and cardiovascular function, in particular, while men and women can equally develop valvular heart disease (VHD), the occurrence of the type of valve disease is typically gender specific. In most cases, women are more likely to suffer from mitral valve diseases, such as MVP or MR, while men are more frequently diagnosed with aortic valve diseases, such as AS and AR. It is important to notice though that women with VHD have been widely underrepresented and undiagnosed. This has led to inaccurate quantification of VHD severity in the female population, as the pathophysiology, clinical manifestation, diagnostic criteria, and management of said diseases are generalized from data obtained on male patients. Left ventricular dimensions and function are distinctly different in healthy women and men, even after taking body size into consideration: women have smaller LV chambers, thus lower stroke volumes, but maintains a similar cardiac output with a higher resting heart rate, higher systolic and diastolic LV elastance (stiffness) at a given age, and aging accentuates these differences, with a steeper increase in LV elastance. Women are also significantly more susceptible (4 times more likely) to idiopathic pulmonary arterial hypertension (PAH). This strongly suggests underlying sex differences in pulmonary vascular function, remodeling, and reactivity.

In an embodiment, due to the significant differences between the various CPD assessments, a different model is trained for each cardiopulmonary murmur to optimize the performance. Furthermore, to standardize and normalize the performance across different patients for different murmurs and to derive a generalizable measure, we incorporate the following, easily obtainable, noninvasive, clinical, and demographic characteristics of the patients: age, height, gender, and weight.

For the exemplary embodiments of the system and method proposed here may be powered by a battery or through energy harvesting.

Some embodiments of the instant invention are directed to a system for non-invasively determining at least one of cardiopulmonary diseases (CPDs) in a subject's heart, using physiological knowledge of the cardiopulmonary mechanics captured by the sensors.

In an alternative embodiment, relevant features are automatically learned from the raw signals without manual feature engineering using machine learning techniques, including deep multimodal representational learning.

The exemplary embodiments of the system and method proposed here provide an analysis algorithm to non-invasively measure a physical property of said subject's heart that is correlated with said subject's heart beat so as to provide timing signals comprising timing information with respect to heartbeat cycles, and wherein said signal processor is configured to receive said peripheral pressure signals and said timing signals and to non-invasively determine an estimated value of said subject's HML, VHDs, etc. based at least partially thereon.

The exemplary embodiments of the system and method proposed here provide an analytics engine that may use one or more artificial intelligence-based algorithms, including trained algorithms such as machine learning algorithms based on neural networks or other similar technologies, to identify new patterns in time series data. For example, a trained artificial intelligence (AI) algorithm may be a trained machine learning algorithm that may be implemented by a deep learning approach.

For the exemplary embodiments of the system and method proposed here, the trained artificial intelligence algorithm may take as input recorded biological sensor data in order to determine a health condition of a patient, and in some examples, determine a specific treatment. For example, the biological sensor data may be input into the trained AI algorithm. Additional information such as age, gender, recording position, weight, or organ type may be inputted into the trained AI algorithm. The trained AI algorithm may output a likelihood of a pathology or disease, a disease severity score, or a healthy state, for example. In some cases, the trained AI algorithm may be used to analyze a subset of biological sensor data, such as audio data, ECG data, or both the audio and the ECG data.

For the exemplary embodiments of the system and method proposed here, as a non-limiting example, a trained AI algorithm may take as input time-synchronized time series data that includes ECG data and cardiovascular and/or pulmonary vibrational data, obtained from an ECG transducer and audio transducer of the monitoring device, process the input ECG and cardiovascular and/or pulmonary vibrational data, and output a cardiovascular and/or pulmonary condition of the patient. During the processing, the AI algorithm may extract features of the input ECG and cardiovascular and/or pulmonary vibrational data and evaluate the extracted features with respect to plurality of annotated data sets comprising time synchronized ECG data, cardiovascular and/or pulmonary vibrational data, wherein the plurality of annotated data sets (labelled by domain experts) are stored in a database of the analytics engine, the plurality of annotated data sets obtained from a plurality of subjects. For example, the features of the vibrational data, the ECG data, or a combination of the vibration and ECG data can be used to determine a cardiovascular and/or pulmonary condition of a subject. In some examples, the trained AI algorithm may compare and identify a number of examples from the annotated data sets that are closest in terms of a plurality of features of ECG data and/or vibrational data to a recorded ECG or vibrational data. The identified datasets that include features highly similar to the recorded data that is currently analyzed may be provided as output to assist the clinician in diagnosis.

For the exemplary embodiments of the system and method proposed here, additionally, the trained artificial intelligence algorithm may analyze the input ECG and cardiovascular and/or pulmonary vibrational data with a plurality of ECG and hear sound data of the same patient obtained at various times prior to the input data to determine progression of the cardiopulmonary condition. The role of the server 103 in handling recording and AI requests is discussed in further detail below in relation to FIG. 3.

For the exemplary embodiments of the system and method proposed here, the trained AI algorithm may be implemented by a deep learning model. Deep learning models have been proven to be very efficient for image and signal analysis, like image classification and segmentation, speech recognition, language translation, and structured data analysis. Deep learning is based on deep neural networks with hierarchical intermediate layers of artificial neurons, which can progressively extract increasingly complex features. By learning very complicated input-output relationships, deep learning can outperform systems that are based on manual feature extraction, like rule-based expert systems. The deep learning model may be trained by a supervised method, on deep convolutional neural networks (CNNs). A training data set for supervised learning problem may biological sensor data reviewed and labeled by domain experts (e.g., physicians, clinicians, etc.). In some examples, the training may be implemented by an unsupervised method or a semi-supervised method based on deep CNNs.

For the exemplary embodiments of the system and method proposed here, the deep learning model may be applicable to any sensory database, image, motion image/video, single or multichannel sensor signals (from sensors such as cardiac waveform sensors, ECG, accelerometers, gyroscopes, acoustic microphones, micro-electro-mechanical systems-MEMS, microwave, radar, radio-frequency, doppler, or Near Field Communication-NFC) and structured data. After proper training with pairs of cardiovascular input-output, it can produce absolute markers, such as but not limited to heart valvular events (e.g., MVO, MVC, AVO, AVC, etc), E/e′, ejection fraction (EF), global longitudinal strain (GLS), left atrial volume index, different cardiac time intervals (CTI's), mean Pulmonary Artery pressures (mPAP), systolic Pulmonary Artery pressures (sPAP), and diastolic Pulmonary Artery pressures (dPAP), Pulmonary Wedge Capillary Pressure (PCWP), Cardiac Output and stroke volume.

The deep learning core of the embodiment of the system and method proposed here, can be deep feed forward (DFF), deep belief network (DBN), deep convolutional network (DCN), deep convolutional inverse graphics network (DCIGN), Deep Boltzmann Machine (DBM), or any other deep structure.

For the exemplary embodiments of the system and method proposed here, the algorithm may be trained by a training set that is specific to a given application, such as, for example classifying a state or condition (e.g., a disease). The training data set for a cardiovascular condition may be different from a training data set for a lung condition, for example. In some examples, the training set (e.g., type of data and size of the training set) may be selected such that, in validation, the algorithm yields an output having a predetermined accuracy, sensitivity and/or specificity (e.g., an accuracy of at least 90% when tested on a validation or test sample independent of the training set).

For the exemplary embodiments of the system and method proposed here, further, during the processing by the trained algorithm, data from different time periods may be compared, whereby a first set of biological data of a patient over a first time period may be processed and/or compared with and a second set of biological data of the patient over a second time period. As a non-limiting example, a patient with aortic stenosis may transmit recorded ECG data to a server over a period of days, months, or years, and for periodic remote consultation, a specialist may process the ECG data recorded over the first year with the ECG data recorded over the second year via a program that uses an expert system or trained algorithm to identify trends, which may determine that the patient's VHD, murmur level, etc., is gradually changing.

For the exemplary embodiments of the system and method proposed here, patient biological data that is recorded and transmitted to the computing system may be processed on its own, and/or along with patient data previously transmitted by the patient, or it may be processed (e.g., compared) with control samples and/or patient data transmitted by other patients. For example, during a single remote consultation with a patient, a remote clinician may run an analytics program on ongoing live stream data, to determine a health condition of the patient. Additionally or alternatively, the patient data is compared with historical data from a variety of anonymized patients, thereby determining that the patient is suffering from a particular kind of cardiopulmonary disease (CPD), and/or the patient's data may be compared with historical data for the patient over the duration of the patient's relationship with the care facility to evaluate a patient's disease progression.

The exemplary embodiment of system and method described is the development on an embedded hardware system, the main elements required to capture body sounds are the sensor unit that captures the body sounds, digitization, and digital processing of the body sounds for noise reduction, filtering and amplification.

It will be apparent to those skilled in the art that various modifications may be made in the present invention without departing from the spirit or scope of the invention. Thus, it is intended that the present invention cover the modifications and variations of this invention provided they come within the scope of the method and system described and their equivalents.

What is claimed is:

1. A method for noninvasive evaluating and monitoring of abnormal cardiovascular or pulmonary vibrations based on quantitative measurements of cardiovascular or pulmonary events for diagnostic and therapeutic purposes, comprising:
obtaining one or more signals using one or more noninvasive cardiopulmonary vibration signal sensors or transducers that provide a measure of one or more physiological effects that are correlated with cardiopulmonary functions;
recording, by at least one processor and the one or more cardiopulmonary vibration signal sensors or transducers for turbulence recordings;
analyzing the turbulence recordings for unique vibration signatures associated with cardiopulmonary disease by searching for one or more deviations from an average beat; and
presenting an assessment of cardiopulmonary disease upon finding that the one or more deviations are above a predetermined threshold.

2. The method of claim 1, wherein the method further comprises the step of seeking a largest mean absolute error of a specific segment between an average beat and each beat from a subject to provide a cardiopulmonary murmur level indicative of cardiopulmonary disease.

3. The method of claim 1, wherein the one or more noninvasive cardiopulmonary vibration signal sensors or transducers are one or more noninvasive seismocardiography (SCG), GCG, PCG, BCG, vibration sensors or transducers.

4. The method of claim 1, wherein a cardiovascular disease evaluated and monitored by the method is coronary artery disease (CAD).

5. The method of claim 1, wherein a cardiopulmonary disease evaluated and monitored by the method is chronic obstructive pulmonary disease (COPD).

6. The method of claim 1, wherein a valve disease or diseases evaluated and monitored is one or more murmurs, among aortic stenosis (AS), mitral regurgitation (MR), aortic regurgitation (AR), mitral stenosis (MS), or mitral valve prolapse (MVP).

7. The method of claim 1, wherein the analyzing of the turbulence recordings occurs between a first heart sound and a second heart sound.

8. The method of claim 7, wherein the first heart sound is S1 and the second heart sound is S2 or the first heart sound is S2 and the second heart sound is S3 or the first heart sound is S3 and the second heart sound is S4.

9. The method of claim 1, wherein analyzing the turbulence recordings for unique vibration signatures associated with cardiopulmonary disease is done by searching for the one or more deviations from the average beat during a late systole segment or during a diastole segment.

10. A system for noninvasive evaluating and monitoring of abnormal vibrations based on quantitative measurements of cardiovascular or pulmonary events for diagnostic and therapeutic purposes, comprising:
at least one or more non-invasive vibration signal sensors or transducers for capturing one or more waveform signals providing a measure of one or more physiological effects that are correlated with cardiopulmonary functions;
one or more processors operatively coupled to the at least one or more non-invasive vibration signal sensors or transducers;
memory having computer instructions and coupled to the one or more processors, the computer instructions which when executed by the one or more processors causes the system to perform the operations of:
obtaining one or more signals using the one or more noninvasive cardiopulmonary vibration signal sensors or transducers that provide the measure of one or more physiological effects that are correlated with cardiopulmonary functions;
recording, by the at least one or more processors and the one or more heart vibration signal sensors or transducers for turbulence recordings between a first cardiac sound and a second cardiac sound;
analyzing the turbulence recordings between the first cardiac sound and the second cardiac sound for unique vibration signatures associated with cardiopulmonary disease by searching for deviations from an average beat; and
presenting an assessment of the cardiovascular or pulmonary disease upon finding that the one or more deviations are above a predetermined threshold.

11. The system of claim 10, wherein the system further comprises the step of seeking a largest mean absolute error of a specific segment between an average beat and each beat from a subject to provide a cardiopulmonary murmur level indicative of cardiopulmonary disease.

12. The system of claim 10, wherein the one or more noninvasive cardiopulmonary vibration signal sensors or transducers comprise at least two sensors comprising one or more noninvasive sensors among an electrocardiogram (ECG) sensor, seismocardiography (SCG) sensor, a gyrocardiography (GCG) sensor, an impedance cardiography (ICG) sensor, a phonocardiophy (PCG) sensor, a photoplethysmogram (PPG) sensor, or a ballistocardiogram (BCG) sensor.

13. The system of claim 10, wherein the abnormal vibrations evaluated and monitored is one among a number of pathological cardiopulmonary murmurs from vessels that supply blood to the heart that includes coronary artery disease (CAD).

14. The system of claim 10, wherein the abnormal vibrations evaluated and monitored is chronic obstructive pulmonary disease (COPD).

15. The system of claim 10, wherein the abnormal vibrations evaluated and monitored is one or more valvular disease murmurs, among aortic stenosis (AS), mitral regurgitation (MR), aortic regurgitation (AR), mitral stenosis (MS), or mitral valve prolapse (MVP).

16. The system of claim 10, wherein the first cardiac sound is S1 and the second cardiac sound is S2 or the first cardiac sound is S2 and the second cardiac sound is S3.

17. The system of claim 10, wherein the assessment of the cardiopulmonary disease provides an identification of the cardiac disease and a severity level of the identified cardiopulmonary disease.

18. The system of claim 10, wherein analyzing the turbulence recordings between the first cardiac sound and the second cardiac sound for unique vibration signatures associated with cardiopulmonary disease is done by searching for the one or more deviations from the average beat during a late systole segment or during a late diastole segment.

19. An apparatus for noninvasive evaluating and monitoring of cardiopulmonary disease based on quantitative measurements of cardiac events for diagnostic and therapeutic purposes, comprising:
at least two or more non-invasive vibration signal sensors or transducers for capturing one or more cardiac waveform signals providing a measure of one or more physiological effects that are correlated with cardiopulmonary functions using a heart murmur level measurement within a given segment;
one or more processors operatively coupled to the at least one or more non-invasive vibration signal sensors or transducers;
memory having computer instructions and coupled to the one or more processors, the computer instructions which when executed by the one or more processors causes the system to perform the operations of:
obtaining one or more signals using the one or more noninvasive heart vibration signal sensors or transducers that provide the measure of one or more physiological effects that are correlated with cardiopulmonary functions;
recording, by the at least one or more processors and the two or more heart vibration signal sensors or transducers for turbulence recordings between a first cardiac sound and a second cardiac sound;
analyzing the turbulence recordings between the first cardiac sound and the second cardiac sound for unique vibration signatures associated with cardiopulmonary disease by searching for deviations from an average beat; and
presenting an assessment of cardiopulmonary disease upon finding that the one or more deviations are beyond a predetermined threshold beyond a normal cardiopulmonary vibration.

20. The apparatus of claim 19, wherein a first non-invasive vibration signal sensor is an accelerometer sensor (SCG, GCG, PCG, BCG) and wherein a second non-invasive vibration signal sensor or transducer is one among an electrocardiogram (ECG), seismocardiography (SCG) sensor, a gyrocardiography (GCG) sensor, an impedance cardiography (ICG) sensor, a phonocardiophy (PCG) sensor, a photoplethysmogram (PPG) sensor, or a ballistocardiogram (BCG) sensor.
